(19) United States
(12) Patent Application Publication
(10) Pub. No.: US 2023/0313323 A1
(43) Pub. Date: Oct. 5, 2023

(54) ASSAYS FOR DETECTING CORONAVIRUS DISEASE 2019 (COVID-19)

(71) Applicant: Ionian Technologies, LLC, Abbott Park, IL (US)

(72) Inventors: Richard Roth, San Diego, CA (US); Jessica Koranda, Abbott Park, IL (US); Andrew Morris, Abbott Park, IL (US); Mario Neto, Abbott Park, IL (US); Ka-Cheung Luk, Abbott Park, IL (US); Matthew Frankel, Abbott Park, IL (US); Klara Abravaya, Abbott Park, IL (US); Dan Toolsie, Abbott Park, IL (US); Gavin Cloherty, Abbott Park, IL (US)

(21) Appl. No.: 17/906,542

(22) PCT Filed: Mar. 26, 2021
(86) PCT No.: PCT/US21/24358

§ 371 Date: Sep. 16, 2022

Related U.S. Application Data
(60) Provisional application No. 63/000,304, filed on Mar. 26, 2020.
(60) Provisional application No. 63/000,971, filed on Mar. 27, 2020.
(60) Provisional application No. 63/004,773, filed on Apr. 3, 2020.
(60) Provisional application No. 63/049,237, filed on Jul. 8, 2020.
(60) Provisional application No. 63/155,599, filed on Mar. 2, 2021.

(51) Int. Cl. C12Q 1/70

(52) U.S. Cl. CPC C12Q 1/701

(57) ABSTRACT

The present disclosure relates to materials and methods for amplifying and detecting 2019-CoV in a sample, comprising a variety of combinations of amplification oligonucleotides and oligonucleotide probes. The disclosure also relates to oligonucleotide sequences, kits, and methods for detecting COVID-19.

### CROSS-REFERENCE TO RELATED APPLICATIONS

This application claims the benefit of U.S. Provisional Application No. 63/000,304, filed Mar. 26, 2020, U.S. Provisional Application No. 63/000,971, filed Mar. 27, 2020, U.S. Provisional Application No. 63/004,773, filed Apr. 3, 2020, U.S. Provisional Application No. 63/049,237, filed Jul. 8, 2020, and U.S. Provisional Application No. 63/155,599, filed Mar. 2, 2021, the content of each of the aforementioned applications is herein incorporated by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,136 Byte ASCII (Text) file named “2021-03-24_38391-601_ SQL_ST25.txt,” created on Mar. 24, 2021.

### FIELD

The present disclosure relates to methods for amplifying target nucleic acid sequences from SARS-CoV-2 and detecting COVID-19.

### BACKGROUND OF THE INVENTION

A novel coronavirus (SARS-CoV-2 (2019-nCoV)) emerged as a human pathogen in China's Hubei province in late 2019, causing fever, severe respiratory illness, and pneumonia. The disease associated with SARS-CoV-2 was named COVID-19. The novel coronavirus is a member of the betacoronavirus genus, closely related to several bat coronaviruses and to severe acute respiratory syndrome coronavirus (SARS-CoV). However, unlike SARS-CoV, SARS-CoV-2 is rapidly transmitted between humans.

As of the end of February 2021, over 100 million cases of COVID-19 were confirmed in over 200 countries, with complications of COVID-19 cited as the cause of death in over 2.5 million individuals.

### SUMMARY OF THE INVENTION

The disclosure provides reagents, including oligonucleotides, for amplifying and detecting coronavirus SARS-CoV-2 in a sample. In some embodiments, a set of oligonucleotides comprises at least one first amplification oligonucleotide, at least one second amplification oligonucleotide, and at least one probe oligonucleotide. The probe oligonucleotide may comprise a detectable label (e.g., a fluorophore). In some embodiments, the set of oligonucleotides is for recombinase-polymerase amplification and detection of SARS-CoV-2 in a sample. In some embodiments, the reagents comprise a group of oligonucleotides comprising one or more sets of oligonucleotides.

In some embodiments, the set of oligonucleotides for recombinase-polymerase amplification and detection of SARS-CoV-2 in a sample comprises: a first amplification oligonucleotide comprising a nucleic acid sequence with at least 70% similarity to SEQ ID NO: 17, a second amplification oligonucleotide comprising a nucleic acid sequence with at least 70% similarity to SEQ ID NO: 19, and a probe oligonucleotide comprising a nucleic acid sequence with at least 70% similarity to SEQ ID NO: 18; or a first amplification oligonucleotide comprising a nucleic acid sequence with at least 70% similarity to SEQ ID NO: 20 or 21, a second amplification oligonucleotide comprising a nucleic acid sequence with at least 70% similarity to SEQ ID NO: 25 or 26, and a probe oligonucleotide comprising a nucleic acid sequence with at least 70% similarity to any of SEQ ID NOs: 22-24; or a combination thereof, wherein each probe oligonucleotide comprises a detectable label. In some embodiments, the first amplification oligonucleotide comprises a nucleic acid sequence with at least 70% similarity to SEQ ID NO: 20, the second amplification oligonucleotide comprises a nucleic acid sequence with at least 70% similarity to SEQ ID NO: 25, and the probe oligonucleotide comprises a nucleic acid sequence with at least 70% similarity to SEQ ID NO: 22 or 23. In some embodiments, the first amplification oligonucleotide comprises a nucleic acid sequence with at least 70% similarity to SEQ ID NO: 21, the second amplification oligonucleotide comprises a nucleic acid sequence with at least 70% similarity to SEQ ID NO: 25 or 26, and the probe oligonucleotide comprises a nucleic acid sequence with at least 70% similarity to SEQ ID NO: 24.

In some embodiments, a group of oligonucleotides for amplifying and detecting SARS-CoV-2 in a sample comprises a first set of oligonucleotides comprising a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6. In some embodiments, the group of oligonucleotides further comprises a second set of oligonucleotides comprising: a first amplification oligonucleotide comprising a nucleic acid sequence with at least 70% similarity to any of SEQ ID NOs: 11 and 15, a second amplification oligonucleotide comprising a nucleic acid sequence with at least 70% similarity to SEQ ID NO: 3 and a probe oligonucleotide comprising a nucleic acid sequence with at least 70% similarity to SEQ ID NO: 5.

The disclosure also provides methods for detecting SARS-CoV-2 in a sample. The sample may comprise a nasal swab or brush, saliva, mucus, blood, serum, plasma, or feces.

In some embodiments, the methods comprise: contacting a sample with a set or group of oligonucleotides as described herein and reagents for amplification; amplifying one or more target SARS-CoV-2 nucleic acid sequences present in the sample using recombinase-polymerase amplification (RPA); hybridizing one or more of the oligonucleotide probes to one or more amplified target SARS-CoV-2 nucleic acid sequences; and detecting hybridization of the one or more probe oligonucleotide sequences to the one or more amplified SARS-CoV-2 target nucleic acid sequences by measuring a signal from the detectable labels. In some embodiments, the methods further comprise contacting the first and second amplification oligonucleotides from the set of oligonucleotides with a recombinase agent. The presence of one or more signals from the detectable label may indicate hybridization of the one or more probe oligonucleotides to the one or more amplified SARS-CoV-2 target nucleic acid sequences.

The reagents for amplification may comprise: a polymerase; a recombinase agent; a recombinase loading protein; a single-strand binding protein; a nicking enzyme; a helicase; a resolvase; an enzyme cofactor; a buffer; deoxyribonucleotide; or ribonucleotide triphosphates; a crowding agent; ATP, an ATP analog, or an ATP generating system; or combinations thereof.

The disclosure further provides kits for detecting SARS-CoV-2 in a sample comprising at least one set of oligonucleotides, any of the oligonucleotides as disclosed herein, reagents for amplifying and detecting nucleic acid sequences, and/or instructions for use.

Other aspects and embodiments of the disclosure will be apparent in light of the following detailed description and accompanying figures.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

FIGS. 1A and 1B are graphs of the Recombinase-Polymerase Amplification (RPA) of SARS-CoV-2 using various combinations of amplification and probe oligonucleotides.

### DETAILED DESCRIPTION OF THE INVENTION

The present disclosure is predicated, at least in part, on the development of a collection of oligonucleotide sequences that facilitate rapid detection of COVID-19.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “first” and “second” are used in this disclosure in their relative sense only. It will be understood that, unless otherwise noted, those terms are used merely as a matter of convenience in the description of one or more of the embodiments. The terms “first” and “second” are only used to distinguish one element from another element, and the scope of the rights of the disclosed technology should not be limited by these terms. For example, a first element may be designated as a second element, and similarly the second element may be designated as the first element.

The term “oligonucleotide,” as used herein, refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values). The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, for example, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

Oligonucleotides can be single-stranded or double-stranded or can contain portions of both double-stranded and single-stranded sequences. The oligonucleotide can be DNA, both genomic and complimentary DNA (cDNA), RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Oligonucleotides can be obtained by chemical synthesis methods or by recombinant methods.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. The section headings used herein are for organizational purposes only and are not to be construed as limiting the described subject matter in any way.

### 1. Amplification and Probe Oligonucleotides

In one embodiment, the oligonucleotides described herein may be used for nucleic acid amplification (e.g., primers) or as probes for nucleic acid hybridization and detection. The terms “primer,” “primer sequence,” “primer oligonucleotide,” and “amplification oligonucleotide” as used herein, refer to an oligonucleotide which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The amplification oligonucleotides of the present disclosure can be of any suitable size, and desirably comprise, consist essentially of, or consist of about 15 to 50 nucleotides, preferably about 20 to 40 nucleotides. The oligonucleotides of the present disclosure can contain additional nucleotides in addition to those described herein. Depending on the type of amplification process employed, the amplification oligonucleotides can include, for example, a nicking enzyme site and a stabilizing region upstream (see, e.g., U.S. Pat. Nos. 9,689,031; 9,617,586; 9,562,264; and 9,562,263, each of which is incorporated herein by reference in its entirety).

The terms “probe,” “probe sequence,” and “probe oligonucleotide,” refer to an oligonucleotide that can selectively hybridize to at least a portion of a target sequence (e.g., a portion of a target sequence that has been amplified) under appropriate hybridization conditions. In general, a probe sequence is identified as being either “complementary” (e.g., complementary to the coding or sense strand (+)), or “reverse complementary” (e.g., complementary to the anti-sense strand (—)). The probes of the present disclosure can be of any suitable size, and desirably comprise, consist essentially of, or consist of about 10-50 nucleotides, preferably about 12-35 nucleotides.

As used herein, the terms “set,” “primer set,” “probe set,” and “primer and probe set,” refer to two or more oligonucleotides which together are capable of priming the amplification of a target sequence or target nucleic acid of interest (e.g., a target sequence within SARS-CoV-2) and/or at least one probe which can detect the target sequence or target nucleic acid. In certain embodiments, the term “set” refers to a pair of oligonucleotides including a first oligonucleotide that hybridizes with the 5′-end of the target sequence or target nucleic acid to be amplified and a second oligonucleotide that hybridizes with the complement of the target sequence or target nucleic acid to be amplified.

The set of oligonucleotides described herein may be used to amplify and detect one or more target SARS-CoV-2 (2019-nCoV) sequences in a sample. The terms “target sequence” and “target nucleic acid” are used interchangeably herein and refer to a specific nucleic acid sequence, the presence or absence of which is to be detected by the disclosed method. In the context of the present disclosure, a target sequence preferably includes a nucleic acid sequence to which one or more oligonucleotides will hybridize and from which amplification will initiate. The target sequence can also include a probe-hybridizing region with which a probe may form a stable hybrid under appropriate amplification conditions. A target sequence may be single-stranded or double-stranded. The target SARS-CoV-2 sequence may be within any portion of the SARS-CoV-2 genome, e.g., the gene encoding the nucleocapsid (N) protein or the gene encoding RNA dependent RNA polymerase (RDRP).

In some embodiments, the set comprises a first amplification oligonucleotide, a second amplification oligonucleotide, and a probe oligonucleotide. In some embodiments, the set comprises a first amplification oligonucleotide comprising a nucleic acid sequence with at least 70% (e.g., 75%., 80%, 85%, 90%, 91%, 92%, 93%, 94%, 95%, 96%, 97%, 98%, 99%, or 100%) similarity to any of SEQ ID NOs: 1 and 10-16, a second amplification oligonucleotide comprising a nucleic acid sequence with at least 70% similarity to SEQ ID NO: 3, and a probe oligonucleotide comprising a nucleic acid sequence with at least 70% similarity to SEQ ID NO: 5; or a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6; or a first amplification oligonucleotide comprising a nucleic acid sequence with at least 70% similarity to SEQ ID NO: 7, a second amplification oligonucleotide comprising a nucleic acid sequence with at least 70% similarity to SEQ ID NO: 8, and a probe oligonucleotide comprising a nucleic acid sequence with at least 70% similarity to SEQ ID NO: 9; or a first amplification oligonucleotide comprising a nucleic acid sequence with at least 70% similarity to SEQ ID NO: 17, a second amplification oligonucleotide comprising a nucleic acid sequence with at least 70% similarity to SEQ ID NO: 19, and a probe oligonucleotide comprising a nucleic acid sequence with at least 70% similarity to SEQ ID NO: 18; or a first amplification oligonucleotide comprising a nucleic acid sequence with at least 70% similarity to SEQ ID NO: 20 or 21, a second amplification oligonucleotide comprising a nucleic acid sequence with at least 70% similarity to SEQ ID NO: 25 or 26, and a probe oligonucleotide comprising a nucleic acid sequence with at least 70% similarity to any of SEQ ID NOs: 22-24; or a combination thereof.

In some embodiment, the set comprises a first amplification oligonucleotide comprising a nucleic acid sequence with at least 70% similarity to any of SEQ ID NOs: 1 and 10-16, a second amplification oligonucleotide comprising a nucleic acid sequence with at least 70% similarity to SEQ ID NO: 3 and a probe oligonucleotide comprising a nucleic acid sequence with at least 70% similarity to SEQ ID NO: 5; and a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6.

In some embodiments, the set comprises a first amplification oligonucleotide comprising a nucleic acid sequence with at least 70% similarity to SEQ ID NO: 20, a second amplification oligonucleotide comprising a nucleic acid sequence with at least 70% similarity to SEQ ID NO: 25, and a probe oligonucleotide comprising a nucleic acid sequence with at least 70% similarity to SEQ ID NO: 22 or 23.

In some embodiments, the set comprises a first amplification oligonucleotide comprising a nucleic acid sequence with at least 70% similarity to SEQ ID NO: 21, a second amplification oligonucleotide comprising a nucleic acid sequence with at least 70% similarity to SEQ ID NO: 25 or 26, and a probe oligonucleotide comprising a nucleic acid sequence with at least 70% similarity to SEQ ID NO: 24.

In some embodiments, the set comprises oligonucleotides for recombinase-polymerase amplification and detection of SARS-CoV-2 (2019-nCoV) in a sample, comprising: a first amplification oligonucleotide comprising a nucleic acid sequence with at least 70% similarity to SEQ ID NO: 17, a second amplification oligonucleotide comprising a nucleic acid sequence with at least 70% similarity to SEQ ID NO: 19, and a probe oligonucleotide comprising a nucleic acid sequence with at least 70% similarity to SEQ ID NO: 18; or a first amplification oligonucleotide comprising a nucleic acid sequence with at least 70% similarity to SEQ ID NO: 20 or 21, a second amplification oligonucleotide comprising a nucleic acid sequence with at least 70% similarity to SEQ ID NO: 25 or 26, and a probe oligonucleotide comprising a nucleic acid sequence with at least 70% similarity to any of SEQ ID NOs: 22-24; or a combination thereof, wherein each probe oligonucleotide comprises a detectable label. In some embodiments, the first amplification oligonucleotide comprises a nucleic acid sequence with at least 70% similarity to SEQ ID NO: 20, the second amplification oligonucleotide comprises a nucleic acid sequence with at least 70% similarity to SEQ ID NO: 25, and the probe oligonucleotide comprises a nucleic acid sequence with at least 70% similarity to SEQ ID NO: 22 or 23. In some embodiments, the first amplification oligonucleotide comprises a nucleic acid sequence with at least 70% similarity to SEQ ID NO: 21, the second amplification oligonucleotide comprises a nucleic acid sequence with at least 70% similarity to SEQ ID NO: 25 or 26, and the probe oligonucleotide comprises a nucleic acid sequence with at least 70% similarity to SEQ ID NO: 24.

In some embodiments, a group of oligonucleotides for amplifying and detecting SARS-CoV-2 (2019-nCoV) in a sample comprises a first set of oligonucleotides comprising: a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6, wherein the probe oligonucleotide comprises a detectable label.

In some embodiments, the group further comprises a second set of oligonucleotides comprising: a first amplification oligonucleotide comprising a nucleic acid sequence with at least 70% similarity to any of SEQ ID NOs: 11 and 15, a second amplification oligonucleotide comprising a nucleic acid sequence with at least 70% similarity to SEQ ID NO: 3, and a probe oligonucleotide comprising a nucleic acid sequence with at least 70% similarity to SEQ ID NO: 5, wherein the probe oligonucleotide comprises a detectable label

| | Sequence 5′ to 3′ | length |
|---|---|---|
| SEQ ID NO: 1 | AAAGAAGAAGGCTGATGAAACT | 22 |
| SEQ ID NO: 5 | CCTTACCGCAGAGACAGAAGAAACAGC | 27 |
| SEQ ID NO: 3 | TGGAGAAATCATCCAAATCTG | 21 |
| SEQ ID NO: 2 | TGCTTAGAATTATGGCCTCACT | 22 |
| SEQ ID NO: 6 | CGTGTTGTAGCTTGTCACACCGTTTCTA | 28 |
| SEQ ID NO: 4 | ATACTTGAGCACACTCATTAG | 21 |
| SEQ ID NO: 7 | CGACTCCACACGGAGTCGCATTGTGCAAACTTT | 33 |
| SEQ ID NO: 8 | CGACTCCACACGGAGTCGTGGGAACACTGTAGA | 33 |
| SEQ ID NO: 9 | CTTTAATGTTTACTGTAGAGAATAAAACATTAAAG | 35 |
| SEQ ID NO: 10 | AAAGAAGAACGCTGATGAAACT | 22 |
| SEQ ID NO: 11 | AAAGAAGAAGCCTGATGAAACT | 22 |
| SEQ ID NO: 12 | AAAGAAGAACGCTGATGAAAC | 21 |
| SEQ ID NO: 13 | GAGCCTAAAAAGGACAAAAAGAAG | 24 |
| SEQ ID NO: 14 | GAGCCTAAATAGGACAAAAAGAAG | 24 |
| SEQ ID NO: 15 | GGCTGATGAAACTCAAGC | 18 |
| SEQ ID NO: 16 | ATTCCCACCAACAGAGCCT | 19 |

| | Sequence | 3′ Mod |
|---|---|---|
| SEQ ID NO: 17 | TAACATGCTTAGAATTATGGCCTCACTTGTTC | |
| SEQ ID NO: 18 | CTTGCTCGCAAACATACAACGTGTTGTAGC[T(FAM)] | Spacer-C3 |
| | [dSpacer]G[T(BHQ-1)]CACACCGTTTCTAT | |
| SEQ ID NO: 19 | TGACCATTTCACTCAATACTTGAGCACACTC | — |
| SEQ ID NO: 20 | AAAGAAGAAGGCTGATGAAACTCAAGCCTTAC | — |
| SEQ ID NO: 21 | GCTGATGAAACTCAAGCCTTACCGCAGAGACA | — |
| SEQ ID NO: 22 | AGCCTTACCGCAGAGACAGAAGAAACAGCAAAC | Spacer-C3 |
| | [T(FAM)][dspacer][T(BHQ-1)]GACTCTTCTTCCTG | |
| SEQ ID NO: 23 | CGCAGAGACAGAAGAAACAGCAAACTGTGAC[T(FAM)] | Spacer-C3 |
| | C[dspacer][T(BHQ-1)]CTTCCTGCTGCAGA | |
| SEQ ID NO: 24 | AGACAGAAGAAACAGCAAACTGTGACTCTTCT[T | Spacer-C3 |
| | (FAM)]C[dspacer][T(BHQ-1)]GCTGCAGATTTGGA | |
| SEQ ID NO: 25 | ACTGCTCATGGATTGTTGCAATTGTTTGGAGA | — |
| SEQ ID NO: 26 | TTGAGTCAGCACTGCTCATGGATTGTTGCAAT | — |

Any of the oligonucleotides described herein may be modified in any suitable manner so as to stabilize or enhance the binding affinity of the oligonucleotide for its target. For example, an oligonucleotide sequence as described herein may comprise one or more modified oligonucleotide. Furthermore, any of the sequences listed which include internal spacers or modifications may be used without the modifications or spacers.

Any of the oligonucleotides described herein may include, for example, spacers, blocking groups, and modified nucleotides. Modified nucleotides are nucleotides or nucleotide triphosphates that differ in composition and/or structure from natural nucleotides and nucleotide triphosphates. Modifications include those naturally occurring that result from modification by enzymes that modify nucleotides, such as methyltransferases. Modified nucleotides also include synthetic or non-naturally occurring nucleotides. For example, modified nucleotides include those with 2′ modifications, such as 2′-O-methyl and 2′-fluoro. Other 2′-modified nucleotides are known in the art and are described in, for example U.S. Pat. No. 9,096,897, which is incorporated herein by reference in its entirety. Modified nucleotides or nucleotide triphosphates used herein may, for example, be modified in such a way that, when the modifications are present on one strand of a double-stranded nucleic acid where there is a restriction endonuclease recognition site, the modified nucleotide or nucleotide triphosphates protect the modified strand against cleavage by restriction enzymes.

Blocking groups or polymerase-arresting molecules are chemical moieties that inhibit target sequence-independent nucleic acid polymerization by the polymerase. The blocking group may render the oligonucleotide capable of binding a target nucleic acid molecule, but incapable of supporting template extension utilizing the detectable oligonucleotide probe as a target. For example, the presence of one or more moieties which does not allow polymerase progression likely causes polymerase arrest in non-nucleic acid backbone additions to the oligonucleotide or through stalling of a replicative polymerase. Oligonucleotides with these moieties may prevent or reduce illegitimate amplification of the probe during the course the amplification reaction. Examples of blocking groups include, for example, alkyl groups, non-nucleotide linkers, phosphorothioate, alkane-diol residues, peptide nucleic acid, and nucleotide derivatives lacking a 3′-OH, including, for example, cordycepin, spacer moieties, damaged DNA bases and the like. Examples of spacers, include, for example, C3 spacers. Spacers may be used, for example, within the oligonucleotide, and also, for example, at the ends to attach other groups, such as, for example, labels.

Any of the oligonucleotide sequences described herein may comprise, consist essentially of, or consist of a complement of any of the sequences disclosed herein. The terms “complement” or “complementary sequence,” as used herein, refer to a nucleic acid sequence that forms a stable duplex with an oligonucleotide described herein via Watson-Crick base pairing rules, and typically shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater identity with the disclosed oligonucleotide. Nucleic acid sequence identity can be determined using any suitable mathematical algorithm or computer software known in the art, such as, for example, CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FASTM, and SSEARCH) (for sequence alignment and sequence similarity searches). Sequence alignment algorithms also are disclosed in, for example, Altschul et al., J. Molecular Biol., 215(3): 403-410 (1990); Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009); Soding, Bioinformatics, 21(7): 951-960 (2005); Altschul et al., Nucleic Acids Res., 25(17): 3389-3402 (1997); and Gusfield, Algorithms on Strings, Trees and Sequences, Cambridge University Press, Cambridge UK (1997), each of which is incorporated herein by reference in its entirety).

The oligonucleotides described herein may be prepared using any suitable method, a variety of which are known in the art (see, for example, Sambrook et al., Molecular Cloning. A Laboratory Manual, 1989, 2. Supp. Ed., Cold Spring Harbour Laboratory Press: New York, N.Y.; M. A. Innis (Ed.), PCR Protocols. A Guide to Methods and Applications, Academic Press: New York, N.Y. (1990); P. Tijssen, Hybridization with Nucleic Acid Probes—Laboratory Techniques in Biochemistry and Molecular Biology (Parts I and II), Elsevier Science (1993); M. A. Innis (Ed.), PCR Strategies, Academic Press: New York, N.Y. (1995); and F. M. Ausubel (Ed.), Short Protocols in Molecular Biology, John Wiley & Sons: Secaucus, N.J. (2002); Narang et al., Meth. Enzymol., 68: 90-98 (1979); Brown et al., Meth. Enzymol., 68: 109-151 (1979); and Belousov et al., Nucleic Acids Res., 25: 3440-3444 (1997), each of which is incorporated herein by reference in its entirety). Oligonucleotide pairs also can be designed using a variety of tools, such as the Primer-BLAST tool provided by the National Center of Biotechnology Information (NCBI). Oligonucleotide synthesis may be performed on oligo synthesizers such as those commercially available from Perkin Elmer/Applied Biosystems, Inc. (Foster City, CA), DuPont (Wilmington, DE), or Milligen (Bedford, MA). Alternatively, oligonucleotides can be custom made and obtained from a variety of commercial sources well-known in the art, including, for example, the Midland Certified Reagent Company (Midland, TX), Eurofins Scientific (Louisville, KY), BioSearch Technologies, Inc. (Novato, CA), and the like. Oligonucleotides may be purified using any suitable method known in the art, such as, for example, native acrylamide gel electrophoresis, anion-exchange HPLC (see, e.g., Pearson et al., J. Chrom., 255: 137-149 (1983), incorporated herein by reference), and reverse phase HPLC (see, e.g., McFarland et al., Nucleic Acids Res., 7: 1067-1080 (1979), incorporated herein by reference).

The sequence of the oligonucleotides can be verified using any suitable sequencing method known in the art, including, but not limited to, chemical degradation (see, e.g., Maxam et al., Methods of Enzymology, 65: 499-560 (1980), incorporated herein by reference), matrix-assisted laser desorption ionization time-of-flight (MALDI-TOF) mass spectrometry (see, e.g., Pieles et al., Nucleic Acids Res., 21: 3191-3196 (1993), incorporated herein by reference), mass spectrometry following a combination of alkaline phosphatase and exonuclease digestions (Wu et al., Anal. Biochem., 290: 347-352 (2001), incorporated herein by reference), and the like.

### 2. Detectable Label

Any one or more of the oligonucleotide sequences described herein may comprise a detectable label, such that the amplification oligonucleotide(s) and/or the probe oligonucleotide can be measured. In one embodiment, each of the probe oligonucleotide sequences described herein comprise a detectable label. The term “detectable label,” as used herein, refers to a moiety or compound that generates a signal which can be measured and whose intensity is related to (e.g., proportional to) the amount of entity bound thereto. Any suitable detectable label that can be conjugated or linked to an oligonucleotide in order to detect binding of the oligonucleotide to a target sequence can be used, many of which are known in the art. In one embodiment, the detectable label may be detected indirectly. Indirectly detectable labels are typically specific binding members used in conjunction with a “conjugate” that is attached or coupled to a directly detectable label. Coupling chemistries for synthesizing such conjugates are well-known in the art and are designed such that the specific binding property of the specific binding member and the detectable property of the label remain intact. As used herein, “specific binding member” and “conjugate” refer to the two members of a binding pair, e.g., two different molecules, where the specific binding member binds specifically to the polynucleotide of the present disclosure, and the “conjugate” specifically binds to the specific binding member. Binding between the two members of the pair is typically chemical or physical in nature. Examples of such binding pairs include, but are not limited to, antigens and antibodies, avidin/streptavidin and biotin, haptens and antibodies specific for haptens, complementary nucleotide sequences, enzyme cofactors/substrates and enzymes, and the like.

Each of the probe oligonucleotide sequences desirably comprises a detectable label. Each of the probes may be labeled with the same detectable label or different detectable labels.

In some embodiments, the detectable label may be directly detected. Such directly detectable labels include, for example, radioisotopes, fluorophores, chemiluminophores, enzymes, colloidal particles, fluorescent microparticles, intercalating dyes (e.g., SYBR Green or ethidium bromide), and the like. In select embodiments, the detectable label may be a fluorophore, such as a fluorescein-family dye, polyhalofluorescein-family dye, hexachlorofluorescein-family dye, coumarin-family dye, rhodamine-family dye, cyanine-family dye, oxazine-family dye, thiazin-family dye, squaraine-family dye, chelated lanthanide-family dye, azo-family dye, triphenylmethane-family dye, or a BODIPY®-family dye. Examples of fluorophores include, but are not limited to, FAM™, CAL-FLUOR®, QUASAR®, HEX™ JOE™ NED™ PET®, ROX™ TAMRA™, TET™, TEXAS RED®, and VIC®. One skilled in the art will appreciate that directly detectable labels may require additional components, such as substrates, triggering reagents, light, and the like, to enable detection of the label. Methods for labeling oligonucleotides, such as probes, are well-known in the art and described in, e.g., L. J. Kricka, Ann. Clin. Biochem., 39: 114-129 (2002); van Gijlswijk et al., Expert Rev. Mol. Diagn., 1: 81-91 (2001); Joos et al., J. Biotechnol., 35: 135-153 (1994); Smith et al., Nucl. Acids Res., 13: 2399-2412 (1985); Connoly et al., Nucl. Acids. Res., 13: 4485-4502 (1985); Broker et al., Nucl. Acids Res., 5: 363-384 (1978); Bayer et al., Methods of Biochem. Analysis, 26: 1-45 (1980); Langer et al., Proc. Natl. Acad. Sci. USA, 78: 6633-6637 (1981); Richardson et al., Nucl. Acids Res., 11: 6167-6184 (1983); Brigati et al., Virol., 126: 32-50 (1983); Tchen et al., Proc. Natl. Acad. Sci. USA, 81: 3466-3470 (1984); Landegent et al., Exp. Cell Res., 15: 61-72 (1984); A. H. Hopman et al., Exp. Cell Res., 169: 357-368 (1987); and Temsamani et al., Mol. Biotechnol., 5: 223-232 (1996), each of which is incorporated herein by reference in its entirety.

In some embodiments, any one or more of the oligonucleotides described herein may also comprise a quencher moiety. When a detectable label (e.g., a fluorophore) and quencher moiety are held in close proximity, such as at the ends of a probe, the quencher moiety prevents detection of a signal (e.g., fluorescence) from the detectable label. When the two moieties are physically separated, the signal becomes detectable. The quencher may be selected from any suitable quencher known in the art, such as, for example, BLACK HOLE QUENCHER® 1 (BHQ-1®), BLACK HOLE QUENCHER® 2 (BHQ-2®), BLACK HOLE QUENCHER® 3 (BHQ-3®), IOWA BLACK® FQ, and IOWA BLACK® RQ. For example, the oligonucleotide probe may comprise a FAM fluorophore, CAL-FLUOR®, or QUASAR fluorophore and a BHQ-1 or BHQ-2 quencher.

The selection of a particular label and labeling technique will depend on several factors, such as the ease and cost of the labeling method, spectral spacing between different detectable labels used, the quality of sample labeling desired, the effects of the detectable moiety on the hybridization reaction (e.g., on the rate and/or efficiency of the hybridization process), the nature of the amplification method used, the nature of the detection system, the nature and intensity of the signal generated by the detectable label, and the like.

### 3. Methods for Amplifying and Detecting SARS-CoV-2 (2019-nCoV)

The present disclosure provides a method for detecting SARS-CoV-2 (2019-nCoV) in a sample. The method comprises: contacting a sample with the set of oligonucleotides disclosed herein and reagents for amplification; amplifying one or more target SARS-CoV-2 nucleic acid sequences present in the sample; hybridizing one or more of the oligonucleotide probes to one or more amplified target SARS-CoV-2 nucleic acid sequences; and detecting hybridization of the one or more probe oligonucleotide sequences to the one or more amplified SARS-CoV-2 target nucleic acid sequences by measuring a signal from the detectable labels. Descriptions of the oligonucleotides set forth herein with respect to the aforementioned set of oligonucleotides also are applicable to the disclosed method.

The sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a nasal swab or brush, or a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

After the sample is obtained from a subject, the sample may be contacted with the set of oligonucleotides comprising amplification oligonucleotides and probes as described herein to form a reaction mixture. The reaction mixture is then placed under amplification conditions. The term “amplification conditions,” as used herein, refers to conditions that promote annealing and/or extension of the amplification oligonucleotides. Such conditions are well-known in the art and depend on the amplification method selected. Amplification conditions encompass all reaction conditions including, but not limited to, temperature and/or temperature cycling, buffer, salt, ionic strength, pH, and the like.

Amplifying a SARS-CoV-2 nucleic acid sequence in the sample can be performed using any suitable nucleic acid sequence amplification method known in the art. In some embodiments, the amplification includes, but is not limited to, polymerase chain reaction (PCR), reverse-transcriptase PCR (RT-PCR), real-time PCR, transcription-mediated amplification (TMA), rolling circle amplification, nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA), and ligase chain reaction (LCR). In some embodiments, amplification of SARS-CoV-2 (2019-nCoV) nucleic acid sequences is performed using isothermal amplification (e.g., RPA or NEAR). In some embodiments, amplification and detection of SARS-CoV-2 nucleic acid sequences is performed using a point of care device (e.g., the ID NOW system (Abbott)).

In some embodiments, amplification of SARS-CoV-2 nucleic acid sequences is performed using real-time PCR. “Real-time PCR,” as used herein, refers to a PCR method in which the accumulation of amplification product is measured as the reaction progresses, in real time, with product quantification after each cycle, in contrast to conventional PCR in which the amplified DNA product is detected in an end-point analysis. Real-time PCR also is known in the art as “quantitative PCR (qPCR).” Real-time detection of PCR products typically involves the use of non-specific fluorescent dyes that intercalate with any double-stranded DNA and sequence-specific fluorescently-labeled DNA probes. Real-time PCR techniques and systems are known in the art (see, e.g., Dorak, M. Tevfik, ed. Real-time PCR. Taylor & Francis (2007); and Fraga et al., “Real-time PCR,” Current protocols essential laboratory techniques: 10-3 (2008), each of which is incorporated herein by reference in its entirety) and are commercially available from a variety of sources (e.g., m2000rt REALTIME™ PCR system (Abbott Molecular, Inc., Des Plaines, IL); CFX Real-Time PCR Detection Systems (Bio-Rad Laboratories, Inc., Hercules, CA); and TAQMAN™ Real-Time PCR System (ThermoFisher Scientific, Waltham, MA)), any of which can be employed in the methods described herein.

The set of oligonucleotides useful for amplification may comprise a first amplification oligonucleotide comprising a nucleic acid sequence with at least 70% similarity to any of SEQ ID NOs: 1 and 10-16, a second amplification oligonucleotide comprising a nucleic acid sequence with at least 70% similarity to SEQ ID NO: 3, and a probe oligonucleotide comprising a nucleic acid sequence with at least 70% similarity to SEQ ID NO: 5 and/or a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6.

In select embodiments, the isothermal amplification methods may rely on nicking and extension reactions, “nicking and extension amplification,” to amplify shorter sequences in a quicker timeframe than traditional amplification reactions. These methods may include, for example, reactions that use only two amplification oligonucleotides, one or two nicking enzymes, and a polymerase, under isothermal conditions. The set of oligonucleotides useful for nicking and extension amplification may comprise a first amplification oligonucleotide comprising a nucleic acid sequence with at least 70% similarity to SEQ ID NO: 7, a second amplification oligonucleotide comprising a nucleic acid sequence with at least 70% similarity to SEQ ID NO: 8, and a probe oligonucleotide comprising a nucleic acid sequence with at least 70% similarity to SEQ ID NO: 9.

In nicking and extension amplification, a target nucleic acid sequence, having a sense and antisense strand, is contacted with a pair of amplification oligonucleotides. The first amplification oligonucleotide comprises a nucleic acid sequence comprising a recognition region at the 3′ end that is complementary to the 3′ end of the target sequence antisense strand, a nicking enzyme site upstream of said recognition region, and a stabilizing region upstream of said nicking enzyme site. The second amplification oligonucleotide comprises a nucleotide sequence comprising a recognition region at the 3′ end that is complementary to the 3′ end of the target sequence sense strand, a nicking enzyme site upstream of said recognition region, and a stabilizing region upstream of said nicking enzyme site. Two nicking enzymes are provided. One nicking enzyme is capable of nicking at the nicking enzyme site of the first amplification oligonucleotide but incapable of nicking within said target sequence. The other nicking enzyme is capable of nicking at the nicking enzyme site of the second amplification oligonucleotide but incapable of nicking within said target sequence. A DNA polymerase is employed under conditions for amplification which involves multiple cycles of extension of the amplification oligonucleotides thereby producing a double-stranded nicking enzyme site which are nicked by the nicking enzymes to produce the amplification product. For example, see U.S. Pat. Nos. 9,689,031; 9,617,586; 9,562,264; and 9,562,263, and U.S. patent application Ser. Nos. 15/467,893; 15/600,951; and 16/243,829, each of which is incorporated herein by reference in its entirety.

In some embodiments, the ID NOW COVID-19 assay uses a nicking enzyme amplification reaction (NEAR), an isothermal nucleic acid amplification technology, to target a highly conserved region of the RdRp genes in SARS-CoV-2 RNA. In some embodiments, the assay system comprises: a sample receiver, containing elution/lysis buffer; a test base, comprising two sealed reaction tubes, each containing a lyophilized pellet; a transfer cartridge for transfer of the eluted sample to the test base; and an ID NOW instrument. The ID NOW COVID-19 assays deliver positive results in as little as five minutes and negative results in 13 minutes, providing rapid COVID-19 results in a wide range of healthcare settings.

In some embodiments, the ID NOW COVID-19 POC assays provide a lower limit of detection (LOD) of 125 copies/mL or less. In silico analyses found 100% homology of all templates and probes to 957 SARS-CoV-2 sequences reported from 45 countries and 21 cities and provinces of China and predicted no significant cross-reactivity to microorganisms causing common respiratory tract illnesses, including other coronaviruses, influenza, RSV, and rhinovirus.

Clinical performance was determined in 30 contrived samples containing known concentrations of SARS-CoV-2 RNA and 30 contrived negative samples. SARS-CoV-2 RNA was detected in all positive samples (positive percent agreement 100% [CI, 88.6-100%]) and none of the negative samples (negative percent agreement 100% [CI, 88.6-100%]).

In select embodiments, amplification of SARS-CoV-2 nucleic acid sequences is performed using Recombinase-Polymerase Amplification (RPA), which relies on the properties of recombinases and related proteins, to invade double-stranded DNA with single stranded homologous DNA permitting sequence specific priming of DNA polymerase reactions.

The set of oligonucleotides useful for RPA may comprise a first amplification oligonucleotide comprising a nucleic acid sequence with at least 70% similarity to SEQ ID NO: 17, a second amplification oligonucleotide comprising a nucleic acid sequence with at least 70% similarity to SEQ ID NO: 19, and a probe oligonucleotide comprising a nucleic acid sequence with at least 70% similarity to SEQ ID NO: 18; or a first amplification oligonucleotide comprising a nucleic acid sequence with at least 70% similarity to SEQ ID NO: 20 or 21, a second amplification oligonucleotide comprising a nucleic acid sequence with at least 70% similarity to SEQ ID NO: 25 or 26, and a probe oligonucleotide comprising a nucleic acid sequence with at least 70% similarity to any of SEQ ID NOs: 22-24.

In some embodiments, wherein the first amplification oligonucleotide comprises a nucleic acid sequence with at least 70% similarity to SEQ ID NO: 20, the second amplification oligonucleotide comprises a nucleic acid sequence with at least 70% similarity to SEQ ID NO: 25, and the probe oligonucleotide comprises a nucleic acid sequence with at least 70% similarity to SEQ ID NO: 22 or 23. In some embodiments, the first amplification oligonucleotide comprises a nucleic acid sequence with at least 70% similarity to SEQ ID NO: 21, the second amplification oligonucleotide comprises a nucleic acid sequence with at least 70% similarity to SEQ ID NO: 25 or 26, and the probe oligonucleotide comprises a nucleic acid sequence with at least 70% similarity to SEQ ID NO: 24.

In RPA, a recombinase agent is contacted with first and second amplification oligonucleotides to form nucleoproteins. These nucleoproteins contact the target sequence to form a first double stranded structure at a first portion of said first strand and form a double stranded structure at a second portion of said second strand so the 3′ ends of said first amplification oligonucleotide and the second amplification oligonucleotide are oriented towards each other on the DNA comprising the target sequence. The 3′ end of the amplification oligonucleotides in the nucleoprotein are extended by DNA polymerases to generate first and second double stranded nucleic acids, and first and second displaced strands of nucleic acid. The steps are repeated until the desired level of amplification is achieved.

Methods and materials useful for RPA of a target nucleic acid sequence are known in the art. See U.S. Pat. Nos. 7,270,981; 8,460,875; 7,399,590; 7,666,598; 8,030,000; 8,426,134; 8,945,845; 9,663,820; 10,329,603; 10,329,602; 8,017,339; 8,574,846; 8,962,255; 10,036,057; 8,071,308; 10,093,908; and 8,637,253, and U.S. patent application Ser. Nos. 15/099,754; 16/442,007; 14/705,150; and Ser. No. 16/155,133, each of which is incorporated herein by reference in its entirety. For example, suitable recombinase agents include the E. coli RecA protein, the T4 uvsX protein, or any homologous protein or protein complex from any phyla. Other non-homologous recombinase agents may be utilized in place of RecA, for example as RecT, or RecO. Suitable recombinase loading proteins may include, for example, T4uvsY, E. coli recO, E. coli recR and derivatives and combinations of these proteins. Suitable single stranded DNA binding proteins may be the E. coli SSB or the T4 gp32 or a derivative or a combination of these proteins. The DNA polymerase may be a eukaryotic or prokaryotic polymerase. Examples of eukaryotic polymerases include pol-α, pol-β, pol-δ, pol-ε and derivatives and combinations thereof. Examples of prokaryotic polymerase include E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof. Other components of RPA include ATP, an ATP analog, or a system for ATP regeneration (convert ADP to ATP). Such system may utilize, for example, phosphocreatine and creatine kinase. The ATP or ATP analog may be any of ATP, ATP-γ-S, ATP-β-S, ddATP or a combination thereof. The RPA reaction may also include a system to regenerate ADP from AMP and a to convert pyrophosphate to phosphate (pyrophosphate). Suitable crowding agents used in RPA include polyethylene glycol (PEG), dextran and ficoll.

Following amplification of one or more SARS-CoV-2 virus nucleic acid sequences present in the sample, the disclosed method may further comprise hybridizing one or more of the probe oligonucleotide sequences disclosed herein to the one or more amplified target SARS-CoV-2 (2019-nCoV) nucleic acid sequences.

Following hybridization of the one or more of the probe oligonucleotide sequences to the one or more amplified target nucleic acid sequences, the method comprises detecting hybridization of the one or more probe oligonucleotide sequences to the one or more amplified target nucleic acid sequences by assessing a signal from each of the detectable labels, whereby (i) the presence of one or more signals indicates hybridization of the one or more probe oligonucleotide sequences to the one or more target SARS-CoV-2 nucleic acid sequences and the presence of SARS-CoV-2 in the sample, and (ii) the absence of signals indicates the absence of SARS-CoV-2 in the sample. Detection of signals from the one or more probe oligonucleotide sequences may be performed using a variety of well-known methodologies, depending on the type of detectable label. For example, detection may be done using solution real-time fluorescence or using a solid surface method.

### 4. Treatment and Monitoring of Subjects Identified as Having SARS-CoV-2

A subject identified according to the methods described herein as having SARS-CoV-2 may be treated, monitored (e.g., for the presence of a SARS-CoV-2 nucleic acid determined in a sample from the subject), treated and monitored, and/or monitored and treated using routine techniques known in the art. In some embodiments, the methods described herein further include treating the subject when the presence of SARS-CoV-2 nucleic acid is determined in one or more samples obtained from the subject using the present methods.

The treatment can take a variety of forms depending on whether the subject is asymptomatic or experiencing mild, moderate, or severe symptoms of SARS-CoV-2 infection. For example, subjects experiencing mild symptoms, will experience a fever, cough (with or without sputum production), anorexia, malaise, muscle pain, sore throat, dyspnea, nasal congestion, headache, diarrhea, nausea, vomiting, or any combination thereof. Subjects experiencing a moderate symptoms will experience a fever greater than 100.4° F. that lasts for several days, chills, shortness of breath, lethargy, or any combination thereof. Such subjects may also suffer from pneumonia. Subjects experiencing severe infection will experience trouble breathing, persistent pain or pressure in the chest, confusion, inability to rouse, bluish lips or face, or any combination thereof. Such subjects may also suffer from severe pneumonia.

If the subject is asymptomatic or has mild symptoms, the subject may be treated with rest, sleep, by keeping warm, ingesting fluids (e.g., remaining hydrated), minimizing social interaction with other subjects (e.g., remain isolated or quarantined, such as, for example, at home), or any combination thereof. Additionally, the subject can be monitored to see if symptoms arise and/or worsen.

Subjects with moderate or severe symptoms of infection with SARS-CoV-2 may be treated with one or more drugs (e.g., remdesivir), vaccines, convalescent plasma therapy (e.g., receiving plasma from blood taken from a subject that has survived an infection with SARS-CoV-2), respiratory support or assistance (e.g., receiving supplemental oxygen through a nasal cannula, face mask, or non-invasive or invasive (e.g., intubation) ventilation) or combinations thereof. Subjects receiving any of the aforementioned treatment can also further be monitored using routine techniques known in the art.

### 5. Vaccinations

In another embodiment, the present disclosure relates to use of the methods described herein in connection with at least one vaccinations and/or re-vaccinations (e.g., further vaccinations) of a subject against SARS-CoV-2 (2019-nCoV). In some embodiments, the present methods are used to detect the presence of a SARS-CoV-2 nucleic acid in at least one sample obtained from a subject to determine whether or not the subject should or can be administered at least one vaccine (e.g., such as a first or initial vaccine, one or more further or additional vaccines, etc.) against SARS-CoV-2. In some embodiments, the subject tested may be naïve, such that the subject does not have any immunity or lacks immunologic immunity to SARS-CoV-2 and was not previously vaccinated against SARS-CoV-3. In some embodiments, the subject may be naïve while have been previously vaccinated against SARS-CoV-2. In some embodiments, the subject may be currently infected with SARS-CoV-2, exhibiting no or mild symptoms, and lacking any previous vaccinations. In some embodiments, the subject may be currently infected with SARS-CoV-2, exhibiting no or mild symptoms, and have been previously vaccinated against SARS-CoV-2. In some embodiments, the subject may have recovered from a previous SARS-CoV-2 infection and have not been previously vaccinated against SARS-CoV-2. In some embodiments, the subject may have recovered from a previous SARS-CoV-2 infection and have been previously vaccinated against SARS-CoV-2.

The above method can be performed regardless of the variation in timing and/or severity of prior infection with a SARS-CoV-2. Using the methods described herein, if the presence of a SARS-CoV-2 nucleic acid is detected in the sample, the subject may need to wait for a period of time (e.g., 30 days, 60 days, 90 days, etc.) to be administered at least one vaccine against SARS-CoV-2; whether the vaccine to be administered is the first dose of the vaccine, a second (e.g., booster) dose of the vaccine, a third or any further additional (e.g., booster) dose of the vaccine. Alternatively, if the presence of a SARS-CoV-2 nucleic acid is not detected in the sample, then at least one vaccine can be administered to the subject.

The phrase “at least one further vaccine or vaccination” or “at least one additional vaccine or vaccination” encompass a scenario wherein a first or current vaccine is administered to a subject followed, at some later period in time, by at least one additional or further vaccine or vaccination (e.g., N+1 (where N is a first or current vaccine plus an additional or further vaccine), N+2 (where N is a first or current vaccine plus two additional vaccines), N+3, N+4, N+5, N+6, N+7, N+8, N+9, N+10 to N+N′ (where N′ is an integer from 1 to 1000, from 1 to 500, from 1 to 100)).

In another embodiment, the methods described herein are used to detect the presence of a SARS-CoV-2 nucleic acid in at least one sample obtained from the subject within a time frame after the subject is administered at least one vaccine for SARS-CoV-2 in order to: determine whether or not the subject should be administered at least one further vaccine (e.g. receive one or more boosters) against SARS-CoV-2; and/or monitor the subject following the administration of at least one vaccine for SARS-CoV-2. In some embodiments, the method involves obtaining the sample within a time frame after the subject has been administered at least one vaccine for SARS-CoV-2. The time frame after the subject has been administered at least one vaccine SARS-CoV-2 can be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, etc. In some embodiments, the sample is obtained within about 7 to about 21 days after the subject has been administered at least one vaccine SARS-CoV-2. Additionally, in yet further embodiments, the monitoring of the subject involves monitoring for post-vaccine symptomology or side-effects (e.g., one or more of fatigue or malaise, headache, dizziness, or lightheadedness, fever or chills, muscle, bone, joint or nerve symptoms, nausea, vomiting, diarrhea, or other digestive symptoms, sleep changes, swollen lymph node, skin/nail or face changes, eye, ear, mouth or throat changes, cough, chest or breathing symptoms and/or memory or mood changes) after a subject receives one or more vaccines (e.g., such as after a first dose of a vaccine for SARS-CoV-2, a second dose of a vaccine for SARS-CoV-2, etc.).

Using the methods described herein, if a SARS-CoV-2 nucleic acid is not detected in the sample then at least one further vaccines (e.g., one or more boosters) can be administered to the subject. Alternatively, if a SARS-CoV-2 nucleic acid is detected in the sample then at least one further vaccine may or may not be administered to the subject.

### 6. Kits

The disclosure also provides a kit for amplifying and detecting SARS-CoV-2 (2019-nCoV) in a sample. The kit comprises at least one oligonucleotide as described herein. In some embodiments, the kit comprises a set or group of oligonucleotides described herein. The kit may further comprise reagents for amplifying and detecting nucleic acid sequences, and instructions for amplifying and detecting SARS-CoV-2. Descriptions of the oligonucleotides and sets of oligonucleotides set forth herein with respect to the aforementioned methods also are applicable to those same aspects of the kit described herein. Many such reagents are described herein or otherwise known in the art and commercially available. Examples of suitable reagents for inclusion in the kit (in addition to the oligonucleotides described herein) include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Other reagents used in amplification reactions include nicking enzymes, single-strand binding proteins, helicases, resolvases, and the like.

The kit may comprise instructions for using the amplification reagents and oligonucleotides described herein, e.g., for processing the test sample, extracting nucleic acid molecules, and/or performing the test; and for interpreting the results obtained. The instructions may be printed or provided electronically (e.g., DVD, CD, or available for viewing or acquiring via internet resources).

The kit may be supplied in a solid (e.g., lyophilized) or liquid form. The various components of the kit of the present disclosure may optionally be contained within different containers (e.g., vial, ampoule, test tube, flask, or bottle) for each individual component (e.g., amplification oligonucleotides, probe oligonucleotides, or buffer). Each component will generally be suitable as aliquoted in its respective container or provided in a concentrated form. Other containers suitable for conducting certain steps of the amplification/detection assay may also be provided. The individual containers are preferably maintained in close confinement for commercial sale.

The kit may further comprise a swab for obtaining a biological sample. In some embodiments, the kit comprises reagents for gaining access to and/or extracting/isolating nucleic acid from a biological sample.

### 7. EXAMPLES

### Example 1

Amplification of SARS-CoV-2 (2019-nCoV) nucleic acid sequences was performed by PCR using various first amplification oligonucleotides with the second amplification oligonucleotide comprising SEQ ID NO: 3 and probe oligonucleotide comprising SEQ ID NO: 5.

The SARS-CoV-2 sample was genomic RNA from SARS-Related Coronavirus 2, Isolate USA-WA1/2020 (BEI Resources). The genomic RNA was extracted from a preparation of cell lysate and supernatant from Cercopithecus aethiops kidney epithelial (Vero E6, ATCC® CRL-1586™) cells infected with SARS-CoV-2, isolate USA-WA1/2020. The viral genomic RNA is in a background of cellular nucleic acid and carrier RNA.

PCR Cycling Parameters:

| | Number | | | |
|---|---|---|---|---|
| | of cycles | Temperature | Time | |
| Reverse Transcription (RT): | 1 | 59° C. | 30 | min |
| Denat/act/deact: | 4 | 95° C. | 40 | s |
| | | 46° C. | 30 | s |
| | 6 | 92° C. | 30 | s |
| | | 60° C. | 30 | s |
| | 37 | 92° C. | 30 | s |

| 56° C. | 20 s + 2 s AU | |
|---|---|---|
| 35° C. | 40 | s |

The PCR reaction mixture included the SARS-CoV-2, a COVID-19 primer set (Primer Set 1) including SEQ ID NO:3 and one of SEQ ID NOs: 1, 10, 12-14, or 16, and a probe oligonucleotide of SEQ ID NO: 5. An internal control nucleic acid and a primer set to the internal control were also included as a positive control where noted. The reaction mixture also included reaction buffers, dNTPs, reference dyes, DNA polymerase (rTth), in concentrations commonly used in the art.

Table 3 shows the results for each of the primer sets. Both the SARS-CoV-2 target and the internal control RNA were amplified under the cycling conditions.

TABLE 3
PCR results for Primer Set 1 with different forward primers

| Rxn | | | SARS-CoV-2 (2019-nCoV) | | | Internal Control (IC) | | |
|---|---|---|---|---|---|---|---|---|
| No. | Primer Set(s) | gE/rxn | Ct* | MR† | dRn‡ | Ct* | MR† | dRn‡ |
| 1 | SEQ ID NOs: 1 and | 1000 | 19.76 | 0.29 | 5.30 | −1.00 | 2.24 | −0.02 |
| | 3; no internal control | | | | | | | |
| 2 | SEQ ID NOs: 1 and | 1000 | 25.78 | 0.06 | 0.65 | 22.18 | 0.25 | 0.37 |
| | 3; internal control | | | | | | | |
| 3 | SEQ ID NOs: 10 and | 1000 | 30.22 | 0.05 | 0.35 | 21.77 | 0.27 | 0.43 |
| | 3; internal control | | | | | | | |
| 4 | SEQ ID NOs: 12 and | 1000 | 21.65 | 0.14 | 2.60 | 21.14 | 0.41 | 0.79 |
| | 3; internal control | | | | | | | |
| 5 | SEQ ID NOs: 13 and | 1000 | 21.16 | 0.24 | 3.73 | 21.20 | 0.49 | 1.67 |
| | 3; internal control | | | | | | | |
| 6 | SEQ ID NOs: 14 and | 1000 | 21.21 | 0.24 | 3.69 | 21.16 | 0.49 | 1.76 |
| | 3; internal control | | | | | | | |
| 7 | SEQ ID NOS: 16 and | 1000 | 21.34 | 0.23 | 3.33 | 21.19 | 0.50 | 1.77 |
| | 3; internal control | | | | | | | |
| 8 | SEQ ID NOs: 1 and | 1000 | 20.41 | 0.19 | 2.59 | 21.23 | 0.44 | 0.77 |
| | 3; alternative internal | | | | | | | |
| | control | | | | | | | |

*Ct = cycle threshold
†MR = maxRatio
‡dRn = reporter value with baseline subtraction

Additional PCR reactions including a second COVID-19 primer set (Primer Set 2) were conducted with a selection of the first COVID-19 primer sets. The second COVID-19 primer set included SEQ ID NO: 2 and SEQ ID NO: 4. The reactions also included SEQ ID NO: 6 as a probe oligonucleotide. As shown in Table 4, Primer Set 2 provided a stronger signal (dRn) compared to any Primer Set 1, but the addition of Primer Set 1 to Primer Set 2 provided the strongest overall signal.

TABLE 4
PCR results for Primer Set 2 with Primer Set 1

| Rxn | | | SARS-CoV-2 (2019-nCoV) | | | Internal Control (IC) | | |
|---|---|---|---|---|---|---|---|---|
| No. | Primer Set(s) | gE/rxn | Ct* | MR† | dRn‡ | Ct* | MR† | dRn‡ |
| 1 | Primer Set 2; internal | 1000 | 19.10 | 0.34 | 7.80 | 20.55 | 0.52 | 1.81 |
| | control | | | | | | | |
| 2 | Primer Set 1 (SEQ ID | 1000 | 18.69 | 0.32 | 8.45 | 20.86 | 0.33 | 0.56 |
| | NOs: 1 and 3); Primer | | | | | | | |
| | Set 2; internal control | | | | | | | |
| 3 | Primer Set 1 (SEQ ID | 1000 | 18.48 | 0.36 | 8.85 | 20.40 | 0.45 | 1.17 |
| | NOs: 13 and 3); Primer | | | | | | | |
| | Set 2; internal control | | | | | | | |
| 4 | Primer Set 1 (SEQ ID | 1000 | 18.93 | 0.37 | 9.14 | 20.54 | 0.51 | 1.64 |
| | NOs: 16 and 3); Primer | | | | | | | |
| | Set 2; internal control | | | | | | | |
| 5 | Primer Set 1 (SEQ ID | 1000 | 18.68 | 0.37 | 7.94 | 20.38 | 0.50 | 1.19 |
| | NOs: 1 and 3); Primer | | | | | | | |
| | Set 2; alternate internal | | | | | | | |
| | control | | | | | | | |
| 6 | Primer Set 2; alternate | 1000 | 19.13 | 0.33 | 7.30 | 20.33 | 0.54 | 1.90 |
| | internal control | | | | | | | |

*Ct = cycle threshold
†MR = maxRatio
‡dRn = reporter value with baseline subtraction

### Example 2

Amplification of SARS-CoV-2 (2019-nCoV) nucleic acid sequences was performed using Recombinase-Polymerase Amplification (RPA) with various combinations of amplification oligonucleotides and probe oligonucleotides. The combinations tested are shown Table 5. Combination 1 was directed to a target region, whereas combinations 2-5 were directed to a second target region.

TABLE 5
Combination of amplification and probe oligonucleotides for RPA

| Combination | | | | Positive |
|---|---|---|---|---|
| No: | FP | RP | Probe | Signal |
| 1 | SEQ ID NO: 17 | SEQ ID NO: 19 | SEQ ID NO: 18 | Y |
| 2 | SEQ ID NO: 20 | SEQ ID NO: 25 | SEQ ID NO: 22 | Y |
| 3 | SEQ ID NO: 20 | SEQ ID NO: 25 | SEQ ID NO: 23 | Y |
| 4 | SEQ ID NO: 21 | SEQ ID NO: 25 | SEQ ID NO: 24 | Y |
| 5 | SEQ ID NO: 21 | SEQ ID NO: 26 | SEQ ID NO: 24 | Y |

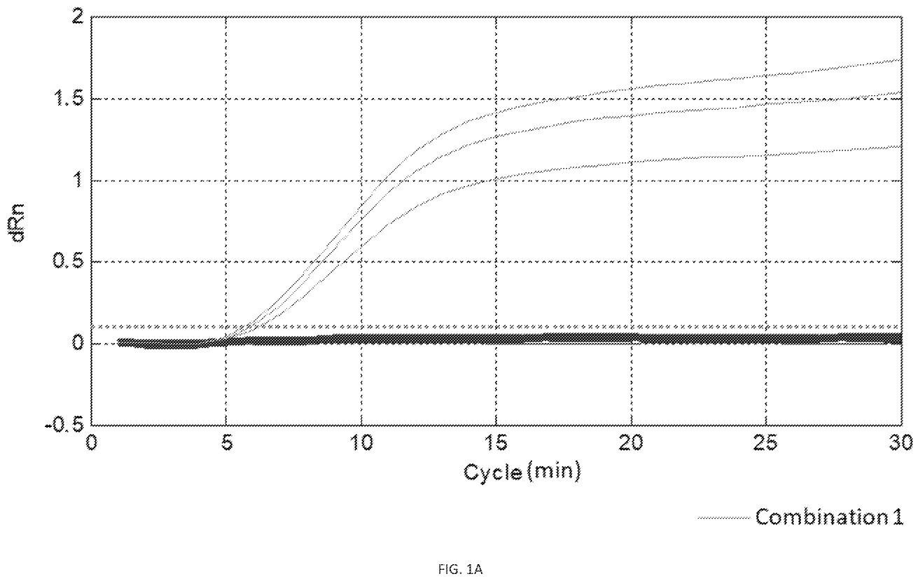

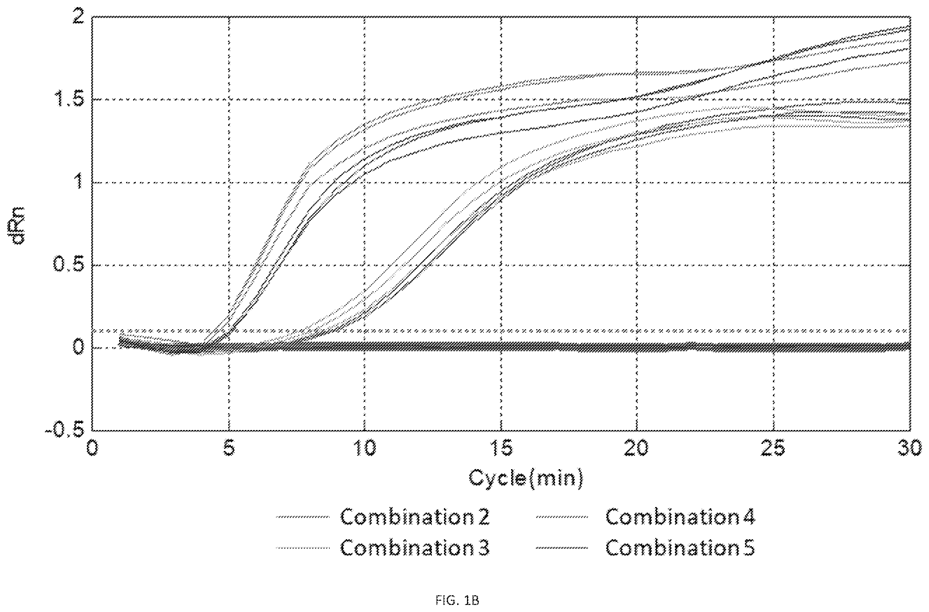

FIGS. 1A and 1B show graphs of the dRn value over time for each of the five combinations. The SARS-CoV-2 target was amplified under the reaction conditions for all of the combinations, with the detectable level of amplification occurring within 10 minutes from the start of the reaction. For combinations 1, 4 and 5, target amplification was detected in 4-6 minutes (FIGS. 1A-1B).

### Example 3

Inclusivity was demonstrated by analyzing the sequence of each of the SARS-CoV-2 primers and probes for homology with all full-length SARS-CoV-2 sequences available in GenBank as of Apr. 28, 2020. A total of 1383 full-length SARS-CoV-2 genome sequences were analyzed from 26 countries/regions (Australia, Brazil, China, Colombia, Czech Republic, France, Greece, Hong Kong, India, Iran, Israel, Italy, Malaysia, Nepal, Netherlands, Pakistan, Peru, South Africa, South Korea, Spain, Sri Lanka, Sweden, Taiwan, Turkey, USA, and Vietnam). 99.5% (1376/1383) exhibited 100% identity to all SARS-CoV-2 primer and probe sequences and 0.5% (7/1383) contained single mismatch against one of the primers or probes.

Inclusivity was further demonstrated by analyzing the sequence of each of the SARS-CoV-2 primers and probes for homology with all full-length SARS-CoV-2 sequences available in the GISAID database as of May 5, 2020. A total of 14,964 full-length SARS-CoV-2 genome sequences were analyzed from 81 countries/regions; 1.1% (170/14,964) contained a single mismatch, 0.04% (6/14,964) contained 2 mismatches, and 0.007% (1/14964) contained 4 mismatches.

### Example 4

Real-time RT-PCR was used to evaluate the disclosed primer sets using known SARS CoV-2 positive, negative, and non-SARS CoV-2 respiratory samples with an Abbott Alinity m System. As shown in Table 6, a dilution series of known positive samples determined a limit of detection of 100 copies/mL. Limit of Detection (LOD) was defined as the lowest detectable concentration of SARS-CoV-2 at which greater than or equal to 95% of all (true positive) replicates test positive.

TABLE 6
LOD of SARS-CoV-2 assay

| Copies/mL | No. positive/No. tested | Avg. CN | % Detected |
|---|---|---|---|
| 400 | 10/10 | 37.52 | 100 |
| 200 | 10/10 | 38.87 | 100 |
| 100 | 19/20 | 39.78 | 95 |
| 50 | 9/10 | 40.20 | 90 |
| 25 | 2/10 | 39.05 | 20 |

109 previously tested frozen Nasopharyngeal swabs were retested in the real-time PCR assay as used above for positive (PPA) and negative (NPA) percent agreement (Table 7). The PPA was found to be 89.8% (53/59) and the NPA was found to be 98% (49/50)

TABLE 7
NPA and PPA of SARS-CoV-2 assay

| | Retest | | |
|---|---|---|---|
| | Positive | Negative | Subtotal |
| Positive | 53 | 6^{a} | 59 |
| Negative | 1^{b} | 49 | 50 |
| Subtotal | 54 | 55 | 109 |

^{a}CN 27.84-31.43
^{b}CN 40.99

Clinical correlation, cross-reactivity, and limit of detection (Table 8) was also evaluated using the Abbott m2000 RealTime SARS-CoV-2 assay.

TABLE 8
m2000 RealTime SARS-CoV-2 Performance Studies

| | | | LOD | Cross- |
|---|---|---|---|---|
| Institution | Comparator | Clinical Correlation | (copies/mL) | Reactivity |
| Univ of | CDC LDA | 96.5% - PPA (28/29) | 100 | None |
| Washington* | | 100% - NPA (30/30) | (93% at 25) | |
| Site 2 | Aldatu LDA | 100% - PPA (20/20) | 100 | None |
| | (PCR) | 100% - NPA (30/30) | | |
| Site 3 | CDC LDA | 100% - PPA (18/18) | 100 | None |
| | | 100% - NPA (15/15) | | |
| Site 4 | Aries (Luminex | 100% - PPA (18/18) | Not | None |
| | PCR) | 95% - NPA (19/20) | completed | |
| Site 5 | CDC LDA | 100% - PPA (10/10) | 100 | None |
| | | 100% - NPA (30/30) | | |
| Site 6 | CDC LDA | 100% - PPA (20/20) | 50 | None |
| | | 100% - NPA (25/25) | | |

*Angeli et al, JVC, Validation and Verification of the Abbott RealTime SARS-CoV-2 Assay Analytical and Clinical Performance, May 2020, incorporated herein by reference.

A total of 104 specimens were analyzed by both Abbott m2000 RealTime SARS-CoV-2 and Alinity m SARS-CoV-2 real-time RT-PCR assays. The positive percent agreement (PPA) between the 2 assays was 100% (47/47) and the negative percent agreement (NPA) was 96.5% (55/57). The results are summarized in Tables 9 & 10.

TABLE 9
Evaluation of Alinity m SARS-CoV-2 assay

RealTime SARS-CoV-2

| | | Positive | Negative |
|---|---|---|---|
| Alinity m | Positive | 47 | 2^{a} |
| SARS-CoV-2 | Negative | 0 | 55 |

^{a}These samples had an Alinity m SARS-CoV-2 CN at 40.99

TABLE 10
NPA and PPA of Alinity m SARS-CoV-2 assay

| | N | Agreement | Exact 95% CI |
|---|---|---|---|
| PPA | 47 | 100% | 92.5, 100.0 |
| NPA | 57 | 96.5% | 87.9, 99.6 |

### Example 5

The SARS-CoV-2 B.1.1.7 strain first identified in the United Kingdom is of utmost concern for evaluation due to the observed link between increased transmissibility and spike gene mutations. While spike gene mutations primarily define the B.1.1.7 lineage, the presence of additional mutations throughout the genome may impact the performance in a variety of diagnostic assays.

Initial in silico examination of B.1.1.7 lineage sequences in GISAID (N=1787, as accessed on Dec. 21, 2020) revealed no lineage-defining mutations of concern for the performance of the primers, probes and methods described herein. A virus culture (BEI NR-54011, EPI_ISL_751801) was heat inactivated at 65° C. for 30 minutes and tested in a dilution series. Multiple dilutions were detected in the expected ranges previously observed with other strains (Table 11). These results confirmed the in silico prediction that the present primers, probes, and methods can reliably detect the B.1.1.7 strain and are consistent with a recent evaluation conducted by Public Health England.

Further evaluation was performed with leftover patient nasopharyngeal swab specimens.

Genome sequences for all specimens were completed and confirmed to belong to the B.1.1.7 lineage. Due to limited remaining volume, leftover VTM (viral transport media) was diluted 5-62.5× before testing. All specimens with sufficient quantity were detected (Table 11).

TABLE 11
SARS-CoV-2 assay results

| Sample ID | Log GE/test* | TCID50/test | Result |
|---|---|---|---|
| BEI | 5.55 | 5.6 | 3/3 Detected |
| BEI | 4.49 | 0.56 | 3/3 Detected |
| BEI | 3.30 | 0.056 | 3/4 Detected |
| BEI | 2.45 | 0.0112 | 0/3 Detected |
| 4 | 4.28 | NA^{#} | Detected |
| 6 | 3.82 | NA^{#} | Detected |
| 13 | 3.41 | NA^{#} | Detected |

^{#}NA, not applicable, TCID₅₀ not determined for patient specimens
*Log genome equivalents (GE)/test were calculated from a standard curve with an R² value of 0.99

The B.1.351 lineage was first identified in South Africa and has since spread to over a dozen countries, with initial reports indicating this variant may escape neutralizing antibodies. The unique mutation profile of the B.1.351 lineage is primarily defined by spike gene mutations K417N, E484K, and N501Y, however, the presence of additional mutations throughout the genome may impact the performance of a variety of diagnostic assays.

Initial in silico examination of B.1.351 lineage sequences in GISAID (N=195, as accessed Dec. 27, 2020) did not reveal any lineage-defining mutations of concern for the performance of the primers, probes and methods described herein. Two virus cultures (BEI NR-54008, NR-54009) were heat inactivated, tested in dilution series, and detected in the ranges previously observed with other strains (Table 12). These results confirmed the in silico prediction that the present primers, probes, and methods can reliably detect the B.1.351 lineage.

TABLE 12
SARS-CoV-2 assay results

| | NR-54008 | | NR-54009 | |
|---|---|---|---|---|
| TCID50/test | Log GE*/test | Result | Log GE*/test | Result |
| 5.6 | 3.94 | 3/3 detected | 3.66 | 3/3 detected |
| 2.8 | 3.64 | 3/3 detected | 3.35 | 3/3 detected |
| 1.4 | 3.36 | 3/3 detected | 3.04 | 3/3 detected |
| 0.56 | 3.00 | 3/3 detected | 2.63 | 2/3 detected |
| 0.28 | 2.76 | 1/3 detected | 2.29 | 0/3 detected |

*Genome equivalents (GE)/test were calculated from a GE/ml standard curve with an R² value of 0.99 and a unit conversion plot of TCID50/mL vs GE/mL with an R² value of 0.97

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A set of oligonucleotides for recombinase-polymerase amplification and detection of SARS-CoV-2 in a sample, comprising:
a first amplification oligonucleotide comprising a nucleic acid sequence with at least 70% similarity to SEQ ID NO: 17, a second amplification oligonucleotide comprising a nucleic acid sequence with at least 70% similarity to SEQ ID NO: 19, and a probe oligonucleotide comprising a nucleic acid sequence with at least 70% similarity to SEQ ID NO: 18; or
a first amplification oligonucleotide comprising a nucleic acid sequence with at least 70% similarity to SEQ ID NO: 20 or 21, a second amplification oligonucleotide comprising a nucleic acid sequence with at least 70% similarity to SEQ ID NO: 25 or 26, and a probe oligonucleotide comprising a nucleic acid sequence with at least 70% similarity to any of SEQ ID NOs: 22-24; or
a combination thereof,
wherein each probe oligonucleotide comprises a detectable label.

2. The set of claim 1, wherein the first amplification oligonucleotide comprises a nucleic acid sequence with at least 70% similarity to SEQ ID NO: 20, the second amplification oligonucleotide comprises a nucleic acid sequence with at least 70% similarity to SEQ ID NO: 25, and the probe oligonucleotide comprises a nucleic acid sequence with at least 70% similarity to SEQ ID NO: 22 or 23.

3. The set of claim 1, wherein the first amplification oligonucleotide comprises a nucleic acid sequence with at least 70% similarity to SEQ ID NO: 21, the second amplification oligonucleotide comprises a nucleic acid sequence with at least 70% similarity to SEQ ID NO: 25 or 26, and the probe oligonucleotide comprises a nucleic acid sequence with at least 70% similarity to SEQ ID NO: 24.

4. The set of claim 1, wherein the detectable label is a fluorophore.

5. A method for detecting SARS-CoV-2 in a sample comprising:
contacting a sample with the set of oligonucleotides of claim 1 and reagents for amplification;
amplifying one or more target SARS-CoV-2 nucleic acid sequences present in the sample using recombinase-polymerase amplification;
hybridizing one or more of the oligonucleotide probes to one or more amplified target SARS-CoV-2 nucleic acid sequences; and
detecting hybridization of the one or more probe oligonucleotide sequences to the one or more amplified SARS-CoV-2 target nucleic acid sequences by measuring a signal from the detectable labels.

6. The method of claim 5, wherein the presence of one or more signals from the detectable label indicates hybridization of the one or more probe oligonucleotides to the one or more amplified SARS-CoV-2 target nucleic acid sequences.

7. The method of claim 5, further comprising contacting the first and second amplification oligonucleotides from the set of oligonucleotides with a recombinase agent.

8. The method of claim 5, wherein the reagents for amplification are selected from the group consisting of: a polymerase; a recombinase; a recombinase loading protein; a single-strand binding protein; a buffer; deoxyribonucleotide; or ribonucleotide triphosphates; a crowding agent; ATP, an ATP analog, or an ATP generating system; or combinations thereof.

9. The method of claim 5, wherein the sample comprises a nasal swab or brush, saliva, mucus, blood, serum, plasma, or feces.

10. A kit for detecting SARS-CoV-2 in a sample comprising:
at least one set of oligonucleotides of claim 1; or
any of oligonucleotides comprising a nucleic acid sequence with at least 70% similarity to SEQ ID NO: 17-26.

11. The kit of claim 10, further comprising reagents for amplifying and detecting nucleic acid sequences, and/or instructions for use.

12. A group of oligonucleotides for amplifying and detecting SARS-CoV-2 in a sample, comprising:
a first set of oligonucleotides comprising: a first amplification oligonucleotide comprising a nucleic acid sequence with at least 70% similarity to SEQ ID NO: 2, a second amplification oligonucleotide comprising a nucleic acid sequence with at least 70% similarity to SEQ ID NO: 4, and a probe oligonucleotide comprising a nucleic acid sequence with at least 70% similarity to SEQ ID NO: 6,
wherein the probe oligonucleotide comprises a detectable label.

13. The group of claim 12, further comprising:
a second set of oligonucleotides comprising: a first amplification oligonucleotide comprising a nucleic acid sequence with at least 70% similarity to any of SEQ ID NOs: 11 and 15, a second amplification oligonucleotide comprising a nucleic acid sequence with at least 70% similarity to SEQ ID NO: 3 and a probe oligonucleotide comprising a nucleic acid sequence with at least 70% similarity to SEQ ID NO: 5,
wherein the probe oligonucleotide comprises a detectable label.

14. The group of claim 12, wherein the detectable label is a fluorophore.

15. A method for detecting SARS-CoV-2 in a sample comprising:
contacting a sample with the group of oligonucleotides of claim 12 and reagents for amplification;
amplifying one or more target SARS-CoV-2 nucleic acid sequences present in the sample;
hybridizing one or more of the oligonucleotide probes to one or more amplified target SARS-CoV-2 nucleic acid sequences; and
detecting hybridization of the one or more probe oligonucleotide sequences to the one or more amplified SARS-CoV-2 target nucleic acid sequences by measuring a signal from the detectable labels.

16. The method of claim 15, wherein the presence of one or more signals from the detectable label indicates hybridization of the one or more probe oligonucleotides to the one or more amplified SARS-CoV-2 target nucleic acid sequences.

17. The method of claim 15, further comprising contacting the first and second amplification oligonucleotides from the set of oligonucleotides with a recombinase agent.

18. The method of claim 15, wherein the reagents for amplification comprise a nicking enzyme, a polymerase, a single-strand binding protein, a recombinase agent, a helicase, a resolvase, an enzyme cofactor, a buffer, deoxyribonucleotide, or ribonucleotide triphosphates, or combinations thereof.

19. The method of claim 15, wherein the sample comprises a nasal swab or brush, saliva, mucus, blood, serum, plasma, or feces.

20. A kit for detecting SARS-CoV-2 in a sample comprising:
at least one group of oligonucleotides of claim 12; or
any of oligonucleotides comprising a nucleic acid sequence with at least 70% similarity to SEQ ID NO: 2-6, 11, and 15.

21. The kit of claim 20, further comprising reagents for amplifying and detecting nucleic acid sequences, and/or instructions for use.


SEQUENCE LISTING (26 sequences)

SEQ ID NO: 1 - DNA, 22 bp, Artificial Sequence
  Synthetic
aaagaagaaggctgatgaaact

SEQ ID NO: 2 - DNA, 22 bp, Artificial Sequence
  Synthetic
tgcttagaattatggcctcact

SEQ ID NO: 3 - DNA, 21 bp, Artificial Sequence
  Synthetic
tggagaaatcatccaaatctg

SEQ ID NO: 4 - DNA, 21 bp, Artificial Sequence
  Synthetic
atacttgagcacactcattag

SEQ ID NO: 5 - DNA, 27 bp, Artificial Sequence
  Synthetic
ccttaccgcagagacagaagaaacagc

SEQ ID NO: 6 - DNA, 28 bp, Artificial Sequence
  Synthetic
cgtgttgtagcttgtcacaccgtttcta

SEQ ID NO: 7 - DNA, 33 bp, Artificial Sequence
  Synthetic
cgactccacacggagtcgcattgtgcaaacttt

SEQ ID NO: 8 - DNA, 33 bp, Artificial Sequence
  Synthetic
cgactccacacggagtcgtgggaacactgtaga

SEQ ID NO: 9 - DNA, 35 bp, Artificial Sequence
  Synthetic
ctttaatgtttactgtagagaataaaacattaaag

SEQ ID NO: 10 - DNA, 22 bp, Artificial Sequence
  Synthetic
aaagaagaacgctgatgaaact

SEQ ID NO: 11 - DNA, 22 bp, Artificial Sequence
  Synthetic
aaagaagaagcctgatgaaact

SEQ ID NO: 12 - DNA, 21 bp, Artificial Sequence
  Synthetic
aaagaagaacgctgatgaaac

SEQ ID NO: 13 - DNA, 24 bp, Artificial Sequence
  Synthetic
gagcctaaaaaggacaaaaagaag

SEQ ID NO: 14 - DNA, 24 bp, Artificial Sequence
  Synthetic
gagcctaaataggacaaaaagaag

SEQ ID NO: 15 - DNA, 18 bp, Artificial Sequence
  Synthetic
ggctgatgaaactcaagc

SEQ ID NO: 16 - DNA, 19 bp, Artificial Sequence
  Synthetic
attcccaccaacagagcct

SEQ ID NO: 17 - DNA, 32 bp, Artificial Sequence
  Synthetic
taacatgcttagaattatggcctcacttgttc

SEQ ID NO: 18 - DNA, 47 bp, Artificial Sequence
  Synthetic
cttgctcgcaaacatacaacgtgttgtagctgtcacaccgtttctat

SEQ ID NO: 19 - DNA, 31 bp, Artificial Sequence
  Synthetic
tgaccatttcactcaatacttgagcacactc

SEQ ID NO: 20 - DNA, 32 bp, Artificial Sequence
  Synthetic
aaagaagaaggctgatgaaactcaagccttac

SEQ ID NO: 21 - DNA, 32 bp, Artificial Sequence
  Synthetic
gctgatgaaactcaagccttaccgcagagaca

SEQ ID NO: 22 - DNA, 49 bp, Artificial Sequence
  Synthetic
agccttaccgcagagacagaagaaacagcaaacttgactcttcttcctg

SEQ ID NO: 23 - DNA, 48 bp, Artificial Sequence
  Synthetic
cgcagagacagaagaaacagcaaactgtgactctcttcctgctgcaga

SEQ ID NO: 24 - DNA, 49 bp, Artificial Sequence
  Synthetic
agacagaagaaacagcaaactgtgactcttcttctgctgcagatttgga

SEQ ID NO: 25 - DNA, 32 bp, Artificial Sequence
  Synthetic
actgctcatggattgttgcaattgtttggaga

SEQ ID NO: 26 - DNA, 32 bp, Artificial Sequence
  Synthetic
ttgagtcagcactgctcatggattgttgcaat